(19) United States
(12) Patent Application Publication
(10) Pub. No.: US 2023/0414955 A1
(43) Pub. Date: Dec. 28, 2023

(54) Closed-Loop Technique to Reduce Electrosensation While Treating a Subject Using Alternating Electric Fields

(71) Applicant: Novocure GmbH, Root D4 (CH)

(72) Inventor: Kristen W. CARLSON, Concord, MA (US)

(73) Assignee: Novocure GmbH, Root D4 (CH)

(21) Appl. No.: 18/214,583

(22) Filed: Jun. 27, 2023

Related U.S. Application Data
(60) Provisional application No. 63/355,871, filed on Jun. 27, 2022.

(51) Int. Cl. A61N 1/40; A61B 5/388

(52) U.S. Cl. CPC A61N 1/40; A61B 5/388

(57) ABSTRACT

A subject can be treated with an alternating electric field (e.g., TTFields) by applying the alternating electric field to a target region, and measuring nerve or muscle activity that is generated by the subject's body in response to the alternating electric field. The course of treatment is then modified based on the measured nerve or muscle activity to reduce or eliminate electrosensation. The nerve or muscle activity can be nerve activity that is measured, e.g., based on an evoked compound action potential (ECAP). In some embodiments, the modification is implemented by adjusting an amplitude or frequency of the alternating electric field. In other embodiments, the modification is implemented by applying an electrical signal to the subject's body, where the electrical signal is configured to reduce electrosensation.

### CROSS REFERENCE TO RELATED APPLICATIONS

This application claims the benefit of U.S. Provisional Application 63/355,871, filed Jun. 27, 2022, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-200 kHz). In the prior art Optune® system, TTFields are delivered to patients via four transducer arrays 21-24 that are placed on the patient's skin in close proximity to a tumor (e.g., as depicted in FIG. 1 for a person with glioblastoma). The transducer arrays 21-24 are arranged in two pairs, and each transducer array is connected via a multi-wire cable to an AC signal generator. The AC signal generator (a) sends an AC current through the anterior/posterior pair of transducer arrays 21, 22 for 1 second, which induces an electric field with a first direction through the tumor; then (b) sends an AC current through the left/right pair of arrays 23, 24 for 1 second, which induces an electric field with a second direction through the tumor; then repeats steps (a) and (b) for the duration of the treatment. Each transducer array 21-24 includes a plurality (e.g., between 9 and 20) of capacitively coupled electrode elements, each of which has an electrically conductive substrate with a dielectric layer disposed thereon.

Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier (BBB) so that, e.g., chemotherapy drugs can reach the brain.

### SUMMARY OF THE INVENTION

One aspect of this application is directed to a first method of treating a target region of a subject's body with an alternating electric field. The first method comprises applying an alternating electric field to the target region during a course of treatment; measuring nerve or muscle activity that is generated by the subject's body in response to the application of the alternating electric field; and modifying the course of treatment based on the measured nerve or muscle activity. The alternating electric field has a frequency between 50 kHz and 1 MHz.

In some instances of the first method, the nerve or muscle activity comprises nerve activity and the nerve activity is measured using a passive array of ECAP electrodes. In some instances of the first method, the nerve or muscle activity comprises muscle activity and the muscle activity is measured using electromyography. In some instances of the first method, the nerve or muscle activity comprises muscle activity and the muscle activity is measured by mechanically sensing muscle twitching.

In some instances of the first method, the modifying comprises adjusting an amplitude of the alternating electric field that is applied to the target region based on the measured nerve or muscle activity. In some instances of the first method, the modifying comprises reducing an amplitude of the alternating electric field that is applied to the target region when the measured nerve or muscle activity indicates that electrosensation is expected. In some instances of the first method, the modifying comprises increasing an amplitude of the alternating electric field that is applied to the target region when the measured nerve or muscle activity indicates that the amplitude can be increased without causing electrosensation. In some instances of the first method, the modifying comprises adjusting a frequency of the alternating electric field that is applied to the target region based on the measured nerve or muscle activity. In some instances of the first method, the modifying comprises increasing a frequency of the alternating electric field that is applied to the target region when the measured nerve or muscle activity indicates that electrosensation is expected.

In some instances of the first method, the modifying comprises applying an electrical signal to the subject's body during each of a plurality of time intervals during the course of treatment. In these instances, the electrical signal is configured to reduce electrosensation when the alternating electric field is applied during the course of treatment, and decisions of when to apply the electrical signal are based on the measured nerve or muscle activity. Optionally, in these instances, the decisions of when to apply the electrical signal are based on when the measured nerve or muscle activity indicates that electrosensation is expected. Optionally, in these instances, the electrical signal comprises a train of at least 10 pulses.

Another aspect of this application is directed to a first apparatus for treating a target region of a subject's body with an alternating electric field. The first apparatus comprises an AC voltage generator having an AC output that operates at a frequency between 50 kHz and 1 MHz and at least one control input, and a controller. The controller is configured to (a) accept signals from at least one sensor that measures nerve or muscle activity that is generated by the subject's body in response to the application of the alternating electric field and (b) modify a course of treatment based on the measured nerve or muscle activity.

In some embodiments of the first apparatus, the at least one sensor comprises a set of ECAP electrodes, and the controller is configured to accept signals that represent nerve activity from the set of ECAP electrodes. In some embodiments of the first apparatus, the at least one sensor comprises a set of electromyography electrodes, and the controller is configured to accept signals that represent muscle activity from the set of electromyography electrodes. In some embodiments of the first apparatus, the at least one sensor comprises an accelerometer, and the controller is configured to accept signals that represent muscle activity from the accelerometer.

Some embodiments of the first apparatus further comprise at least one first electrode element configured for positioning on or in the subject's body and at least one second electrode element configured for positioning on or in the subject's body. In these embodiments, the AC output is applied between the at least one first electrode element and the at least one second electrode element.

In some embodiments of the first apparatus, the controller is programmed to send signals to the at least one control input that cause the AC voltage generator to adjust an amplitude of the AC output based on the measured nerve or muscle activity. In some embodiments of the first apparatus, the controller is programmed to send signals to the at least one control input that cause the AC voltage generator to reduce an amplitude of the AC output when the measured nerve or muscle activity indicates that electrosensation is expected. In some embodiments of the first apparatus, the controller is programmed to send signals to the at least one control input that cause the AC voltage generator to increase an amplitude of the AC output when the measured nerve or muscle activity indicates that the amplitude can be increased without causing electrosensation. In some embodiments of the first apparatus, the controller is programmed to send signals to the at least one control input that cause the AC voltage generator to adjust a frequency of the AC output based on the measured nerve or muscle activity. In some embodiments of the first apparatus, the controller is programmed to send signals to the at least one control input that cause the AC voltage generator to reduce a frequency of the AC output when the measured nerve or muscle activity indicates that electrosensation is expected.

Some embodiments of the first apparatus further comprise a signal generator that generates an electrical signal configured to reduce electrosensation when the alternating electric field is applied to the subject's body. In these embodiments, the controller is programmed to activate the signal generator based on the measured nerve or muscle activity.

Some embodiments of the first apparatus further comprise a signal generator that generates an electrical signal configured to reduce electrosensation when the alternating electric field is applied to the subject's body. In these embodiments, the controller is programmed to activate the signal generator based on the measured nerve or muscle activity, and a decision to activate the signal generator is based on when the measured nerve or muscle activity indicates that electrosensation is expected. Optionally, in these embodiments, the electrical signal comprises a train of at least 10 pulses.

Some embodiments of the first apparatus further comprise a signal generator that generates an electrical signal configured to reduce electrosensation when the alternating electric field is applied to the subject's body; at least one first electrode element configured for positioning on or in the subject's body; at least one second electrode element configured for positioning on or in the subject's body; a third electrode element configured for positioning on or in the subject's body; and a fourth electrode element configured for positioning on or in the subject's body. In these embodiments, the controller is programmed to activate the signal generator based on the measured nerve or muscle activity. The AC output is applied between the at least one first electrode element and the at least one second electrode element. And the electrical signal is applied between the third electrode element and the fourth electrode element.

### BRIEF DESCRIPTION OF THE DRAWINGS

FIG. 1 depicts how transducer arrays are positioned for treating glioblastoma using alternating electric fields.

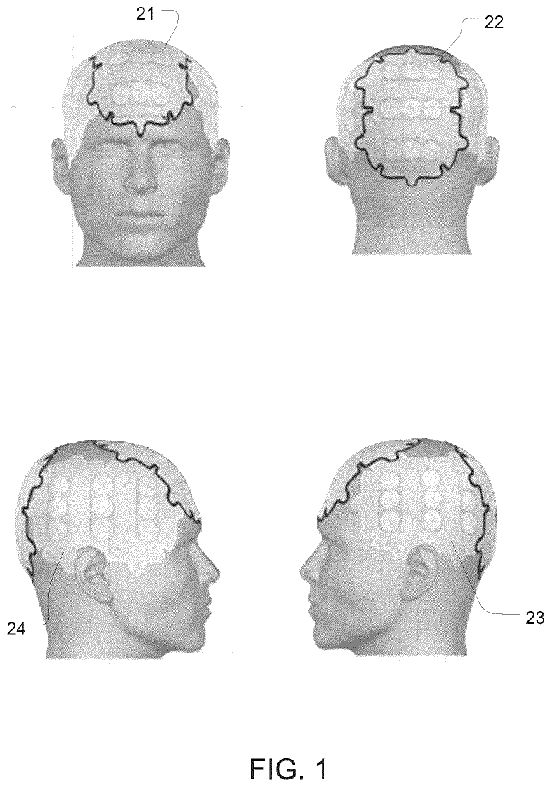

FIG. 2 depicts an apparatus for treating a target region of a subject's body with an alternating electric field that reduces or eliminates electrosensation.

FIG. 3 depicts a method of treating a target region of a subject's body with an alternating electric field.

FIG. 4 depicts another apparatus for treating a target region of a subject's body with an alternating electric field that reduces or eliminates electrosensation.

FIG. 5 depicts another method of treating a target region of a subject's body with an alternating electric field.

Various embodiments are described in detail below with reference to the accompanying drawings, wherein like reference numerals represent like elements.

### DESCRIPTION OF THE PREFERRED EMBODIMENTS

When treating a subject using alternating electric fields, higher amplitudes are strongly associated with higher efficacy of treatment. However, as the amplitude of the alternating electric field increases, and/or as the frequency of the alternating electric field decreases (e.g., to the vicinity of 100 kHz), some subjects experience an electrosensation effect when the alternating electric field switches direction. This electrosensation could be, for example, a vibratory sensation, paresthesia, and/or a twitching or contraction sensation of muscle fibers, or a flicker of light in the eyes (phosphene). And these sensations may discourage some subjects from continuing their treatment using alternating electric fields. The electrosensation is believed to originate from interactions between the alternating electric fields and nerve cells or fibers (i.e., neurons or axons) that are positioned near or adjacent to the transducer arrays.

The embodiments described below determine when electrosensation is either occurring or imminent based on measured nerve or muscle activity that is generated by the subject body in response to the application of the alternating electric fields. The course of treatment is then modified to ameliorate the electrosensation based on the measured nerve or muscle activity.

Some approaches for determining that electrosensation is either occurring or imminent are based on measured nerve activity. During certain types of electrical stimulation of biological tissue, the electrically evoked compound action potential (ECAP) represents the approximately synchronous firing of a population of electrically stimulated nerve fibers. Upon the application of an electrical signal of sufficient energy to activate nerve fibers, fibers of different diameters and in different locations are activated at roughly the same time (e.g., within fractions of milliseconds) and their action potentials (APs) propagate at different velocities to the vicinity of a recording electrode. Further, different nerve fibers of different diameters, which have different activation thresholds and conduction velocities, convey different signals, e.g., of types of sensation (vibration, temperatures, hair movement, muscle contraction, joint position, etc.).

It turns out that the ECAP associated with electrosensation can be measured using a set of electrodes positioned on a subject's skin. These electrodes detect the compounded sum of the individual APs arriving at approximately the same time, which appear as a curve of a given amplitude and duration.

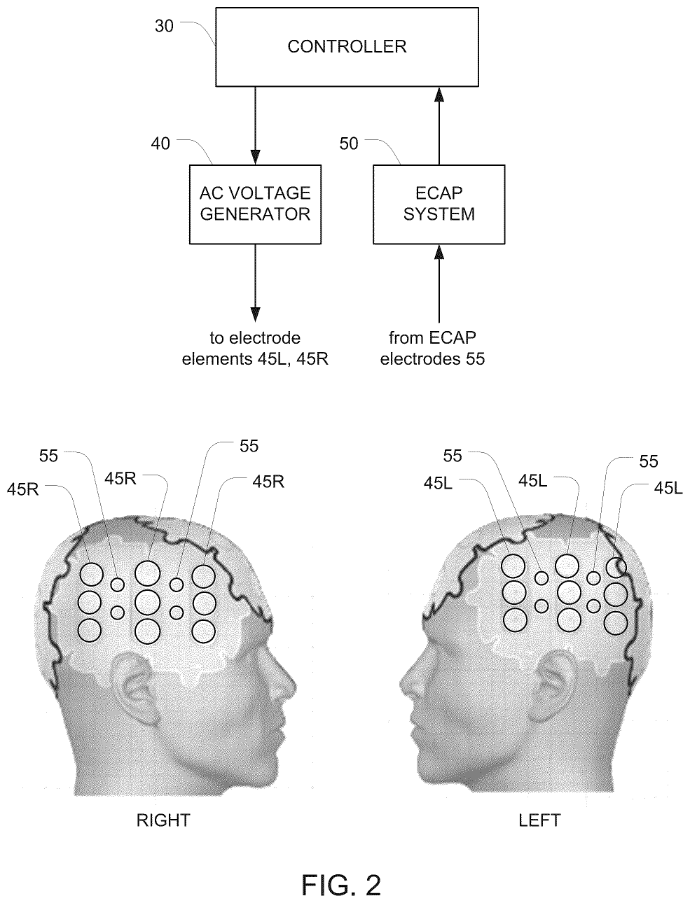

The embodiments described below in connection with FIG. 2-5 rely on ECAP signals that are received using such electrodes to detect or predict whether the subject is probably experiencing electrosensation and/or is close to a threshold beyond which electrosensation is expected, and to modify the course of treatment to ameliorate the electrosensation. One way to modify the course of treatment is to adjust the amplitude of the alternating electric field to ameliorate the electrosensation. Another way to modify the course of treatment is to adjust the frequency of the alternating electric field.

Yet another way to modify the course of treatment is to apply signals which are designed to block the activation of fibers producing electrosensation. A signal that increases the threshold of the electrosensation-signaling nerves, for instance, allows higher amplitudes of the treatment signals to be applied without causing electrosensation. Blocking the propagation of electrosensation signals before they reach the brain will also allow higher treatment amplitudes to be used.

The embodiments described below in connection with FIG. 2-5 use ECAP as feedback to modify the alternating electric fields e.g., for increasing treatment efficacy or reducing undesirable side effects, i.e., ‘closing the loop’ of the treatment process. The closed-loop techniques described herein can also be referred to as adaptive control techniques or adaptive delivery techniques.

FIG. 2 depicts an apparatus for treating a target region of a subject's body with an alternating electric field that avoids or ameliorates electrosensation by using ECAP to measure nerve activity, and modifying the course of treatment based on the measured nerve activity. The FIG. 2 embodiment includes an AC voltage generator 40 that generates AC outputs at a frequency between 50 kHz and 1 MHz (e.g., 50-500 kHz, 75-300 kHz, or 150-250 kHz). The AC voltage generator 40 has at least one control input which may be used, for example, to control the output amplitude of the AC voltage generator 40. The frequency of the AC voltage generator 40 will depend on the type of treatment. For example, to treat a tumor using TTFields, the frequency could be between 150 and 200 kHz. Alternatively, to increase the permeability of a subject's blood-brain barrier, the frequency could be between kHz and 200 kHz (e.g., 100 kHz).

In the example depicted in FIG. 2, a set of first electrode elements 45L is positioned on the subject's body (e.g., on shaved skin) to the left of a target region, and a set of second electrode elements 45R is positioned on the subject's body to the right of the target region. In alternative embodiments, the first and second sets of electrode elements 45L/45R could be implanted in the subject's body (e.g., just beneath the skin) to the left and right of the target region, respectively. When the AC voltage generator 40 applies a voltage between the electrode elements 45L and the electrode elements 45R, an alternating electric field is induced through the target region with field lines that run generally from right to left. The frequency of the alternating electric field will match the frequency of the AC voltage generator 40. The electrode elements 45L/45R can be capacitively-coupled electrode elements or conductive electrode elements.

In addition to the electrode elements 45L/45R which are used to induce the alternating electric field in the target region, independent sets of electrodes 55 are also provided to determine whether the subject is probably experiencing electrosensation or that electrosensation is imminent. More specifically, a first set of ECAP electrodes 55 configured for picking up ECAP signals is positioned near the set of first electrodes 45L, and a second set of ECAP electrodes 55 configured for picking up ECAP signals is positioned near the set of second electrodes 45R. The first and second sets of ECAP electrodes 55 positioned on the left and right sides, respectively, could each be a passive array of electrodes.

Signals from these ECAP electrodes 55 (which can be, e.g., on the order of mV) are accepted by the ECAP measurement system 50, and the ECAP measurement system 50 measures the ECAP on the left and right sides of the subject's body based on signals that arrive from the ECAP electrodes 55 positioned on the left and right sides, respectively. The ECAP measurement system 50 processes those signals (e.g., using an amplifier and an analog to digital converter) and forwards the resulting data to the controller 30. In this way, the ECAP that is generated by each side of the subject's body in response to the application of the alternating electric field is measured.

Because the ECAP associated with electrosensation is measured using the ECAP electrodes 55 and the ECAP measurement system 50, and those measurements are reported to the controller 30, the controller 30 can determine whether the subject is probably experiencing electrosensation and/or is close to a threshold beyond which electrosensation is expected. The controller 30 can then modify the course of treatment based on the measured ECAP. The modification based on the measured ECAP could be, for example, adjusting an amplitude or frequency of the alternating electric field that is applied to the target region based on the measured ECAP.

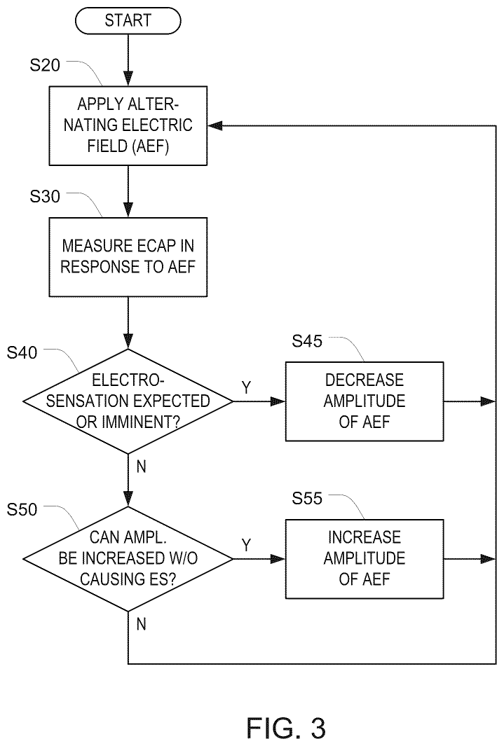

FIG. 3 depicts one example of a method of treating a target region of a subject's body with an alternating electric field, in which the controller 30 modifies the course of treatment based on the measured ECAP. In S20, alternating electric fields are applied to the target region during the course of treatment (e.g., when the AC voltage generator 40 applies an AC output to electrodes 45L/45R). In S30, the evoked compound action potential (ECAP) that is generated by the subject's body in response to the application of the alternating electric field in S20 is measured. This measurement is implemented using the ECAP electrodes 55 and the ECAP measurement system 50 as described above, and the measurements are reported to the controller 30. In S40, the controller 30 ascertains, based on the measured ECAP, if electrosensation is expected. If electrosensation is expected, the method continues at S45, in which the controller 30 sends commands to the control input of the AC voltage generator 40, and those commands cause the AC voltage 40 generator to reduce its output amplitude. The reduction in amplitude will ameliorate the electrosensation.

If electrosensation is not expected, the method proceeds to S50, where the controller 30 determines, based on the measured ECAP, if the amplitude can be increased without causing electrosensation. If the result is yes, the method continues at S55, in which the controller 30 issues commands to the AC voltage generator 40 that cause the AC voltage generator to increase its output amplitude (provided that this will not cause any of the electrodes 45 to overheat). This increase in amplitude will improve the efficacy of the alternating electric field treatment without causing discomfort to the subject. If the result in S50 is no, the method of FIG. 3 restarts from the beginning.

In many anatomic locations, it is preferable to use an electric field whose orientation alternates between different directions. In these locations, additional sets of electrode elements 45 (not shown in FIG. 2) may be positioned on other sides (e.g., anterior and posterior) of the target region. In these embodiments, the AC voltage generator 40 is preferably configured to repeatedly alternate between (a) applying a voltage between the left and right electrode elements 45L/45R, and (b) applying a voltage between the anterior and posterior electrode elements. The AC voltage generator 40 can switch between these two states every 1 second, or at a different interval (e.g., between 50 ms and 10 s). The orientation of the electric field in these embodiments will therefore repeatedly alternate back and forth between the left/right and anterior/posterior directions.

In these embodiments where additional sets of electrode elements 45 are positioned on other sides of the target region, corresponding additional sets of ECAP electrodes 55 should be positioned in the vicinity of the additional electrode elements 45 to determine whether the subject is probably experiencing electrosensation or that electrosensation is imminent. The controller 30 processes signals from these additional sets of ECAP electrodes 55 as described above for the left and right sets of ECAP electrodes 55 (e.g., by reducing the amplitude of the voltage that is being applied to the additional sets of electrode elements 45 when electrosensation is expected at those electrode elements 45).

Adjusting the amplitude of the output of the AC voltage generator 40 (as described above in connection with FIGS. 2-3) is not the only way to avoid or ameliorate electrosensation. To the contrary, because lower frequencies typically cause more electrosensation than higher frequencies, another way to avoid or ameliorate electrosensation is for the controller 30 to send commands to the AC voltage generator 40 that increase the frequency of the alternating electric field whenever the controller 30 determines that electrosensation is occurring or is imminent.

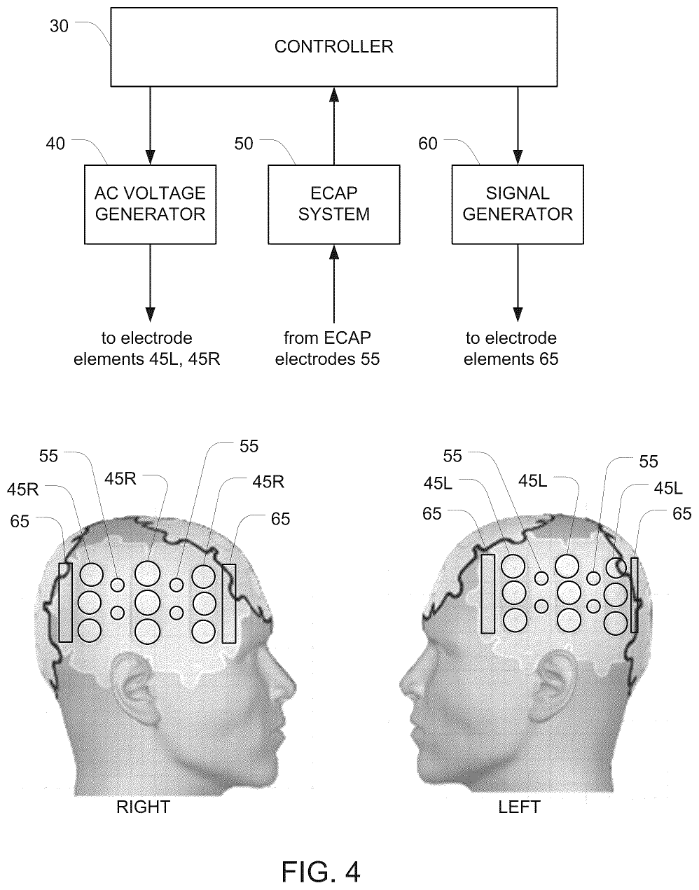

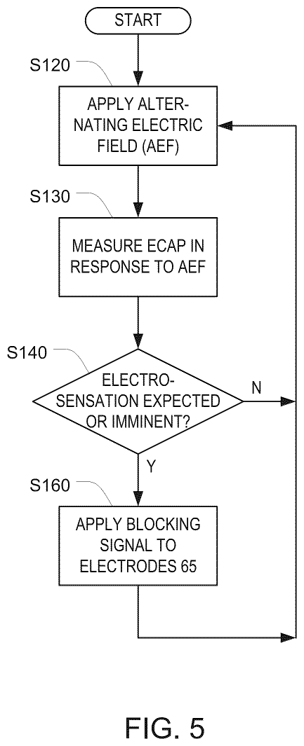

FIGS. 4-5 depict another approach for treating a target region of a subject's body with an alternating electric field that avoids or ameliorates electrosensation by using ECAP to measure nerve activity. Notably, these embodiments avoid or ameliorate electrosensation by applying additional electrical signals to the subject's body. These additional electrical signals are configured to reduce the subject's sensation when the alternating electric field is applied during the course of the treatment.

As noted above, electrosensation is believed to originate from interactions between the alternating electric fields and nerve cells or fibers (i.e., neurons or axons) that are positioned near or adjacent to the transducer arrays. Without being bound by this theory, the additional electrical signals that are applied in the FIGS. 4-5 embodiments are believed to reduce the subject's sensation by increasing the action potential threshold of the relevant nerve cells or otherwise blocking their activation, such as by interfering with the operation of ion channel gates or obstructing AP propagation through similar effects on axon regions near the point of AP generation.

The electrical signal that reduces the subject's sensation may comprise a train of at least 10 pulses. In some embodiments each such electrical signal may comprise of train of at least 12 pulses, at least 15 pulses, or at least 20 pulses, since experiments have shown that different nerve fibers respond to different blocking signal designs. For example, the activation threshold of median and sural nerves has been shown to increase significantly in response to trains of 10 or more pulses, but not to a single pulse. In some embodiments each electrical signal may comprise a train of 10 to 15 pulses or a train of 10 to 20 pulses. In some embodiments, each of these pulses has a width of at least 100 μs. In some embodiments each of these pulses has a width of at least 150 μs, 200 μs, 250 μs, 300 μs, or 400 μs. In some embodiments each of these pulses has a width of 100 μs to 500 μs, 100 μs to 250 μs, or 100 μs to 200 μs. In some embodiments each of these pulses has a width of at least 20 ms, 50 ms, or 100 ms. In some embodiments each of these pulses has a width of 20-50 ms, 50-100 ms, or 100-200 ms.

In some embodiments, the train of pulses continues for at least 100 ms. In some embodiments, the train of pulses continues for at least 150 ms, 200 ms, 250 ms, 300 ms, or 400 ms. In some embodiments, the train of pulses continues for 100 ms to 500 ms, 100 to 250 ms, or 100 to 200 ms. In some embodiments, the pulses are configured to provide a charge balanced waveform.

The threshold increase of median and sural nerves has been shown to be sustained for longer periods of time when the applied blocking trains last for several hundred milliseconds, and for some blocking protocols the threshold increase can last for a few minutes or even tens of minutes. Longer periods of threshold increase are desirable since less frequent applications of the blocking signals are necessary, simplifying treatment and reducing energy requirements.

The electrical signal that reduces the subject's sensation can also have a frequency between 4 kHz and 30 kHz. Alternatively, the electrical signal can have a frequency between 0.1 and 30 Hz (e.g., 0.1-1 Hz or 1-10 Hz). In some embodiments, the electrical signal has a frequency between 1 and 2 Hz. In some embodiments, the electrical signal has an amplitude of 0.5-10 mA. In some embodiments, the amplitude is 0.5 to 1 mA, 1 to 2 mA, or 2 to 10 mA. In some embodiments, the electrical signal has a duration between 1 and 60 s. In some embodiments, the electrical signal has a duration of less than 10 s (e.g., between 1 and 10 s, between 1 and 2 s, between 2 and 5 s, or between 5 and 10 s).

The electrical signal that reduces the subject's sensation can also have a frequency between 0.25 and 10 Hz (e.g., between 0.5 and 5 Hz, or between 1 and 2 Hz). In these embodiments, the electrical signal may optionally be offset from zero amplitude. In these embodiments, each of the electrical signals may have a duration between 100 ms and 30 s, between 200 ms and 20 s, between 500 ms and 20 s, or between 500 ms and 10 s.

The electrical signal that reduces the subject's sensation can be below a threshold of nerve fibers that produces unwanted sensation, or can be above that threshold. In some embodiments, the electrical signals are initially applied below the threshold of nerve fibers that produce unwanted sensation, and after the initial electrical signals have caused an increase in the action potential threshold of the relevant nerve cells, their amplitude is subsequently increased to above that threshold. The electrical signals that are applied may be below a threshold of 7-15 μm Abeta nerve fibers that produces sensations of at least one of vibration and paresthesia, or may be above that threshold. In some embodiments, the electrical signals are initially applied below the threshold of 7-15 μm Abeta nerve fibers that produces sensations of at least one of vibration and paresthesia, and after the initial electrical signals have caused an increase in the action potential threshold of the relevant nerve cells, their amplitude is subsequently increased to above their original threshold. The electrical signal is preferably always below a threshold of nerve fibers that produces at least one of muscle twitching and contraction.

FIG. 4 depicts another apparatus for treating a target region of a subject's body with an alternating electric field that relies on ECAP to reduce or eliminate electrosensation. The FIG. 4 embodiment includes an AC voltage generator 40 that is similar to the AC voltage generator 40 described above in connection with FIG. 2. The frequency of the AC voltage generator 40 will depend on the type of treatment, as described above in connection with FIG. 2.

The FIG. 4 embodiment includes a set of first electrode elements 45L and a set of second electrode elements 45R, which are used to induce the alternating electric field in the target region and are similar to the correspondingly-numbered electrode elements described above in connection with FIG. 2. In addition, the FIG. 4 embodiment has a plurality of sets of ECAP electrodes 55 that are similar to the ECAP electrodes 55 described above in connection with FIG. 2. Signals from these ECAP electrodes 55 are accepted and processed by the ECAP measurement system 50 and resulting data is forwarded to the controller 30 (e.g., as described above in connection with FIG. 2).

Because the ECAP associated with electrosensation is measured using the ECAP electrodes 55 and the ECAP measurement system 50, and those measurements are reported to the controller 30, the controller 30 can determine whether the subject is probably experiencing electrosensation and/or is close to a threshold beyond which electrosensation is expected. The controller 30 can then modify the course of treatment based on the measured ECAP.

In this FIG. 4 embodiment, the course of treatment is modified by using a signal generator 60 to apply an electrical signal to the subject's body via yet another set of electrode elements 65 during each of a plurality of time intervals during the course of treatment. The electrical signal that is generated by the signal generator 60 is configured to reduce electrosensation when the alternating electric field is applied to the subject's body during the course of treatment (e.g., as described above).

In the example illustrated in FIG. 4, the electrode elements 65 are arranged in pairs, and are positioned adjacent to and on opposite sides of the respective set of electrode elements 45L/45R. As a result, when an electrical signal is applied between the electrode elements 65, the electrical signal will traverse the area of skin beneath electrode elements 45L/45R. The electrical signal will interact with nerve fibers in those regions, and this interaction will reduce electrosensation during times that the electrode elements 45L/45R are active. Note that, as used herein, adjacent means nearby; and a touching or abutting relationship is not required by the word adjacent.

The controller 30 decides when the signal generator 60 should apply the electrical signal to the electrode elements 65 based on the measured ECAP, and implements that decision by sending appropriate commands to the signal generator 60. For example, the decision of when to apply the electrical signal can be based on when the measured ECAP (which is measured using the ECAP electrodes 55 and the ECAP system 50) indicates that electrosensation is expected.

FIG. 5 depicts one example of a method of treating a target region of a subject's body with an alternating electric field, in which the controller 30 of FIG. 4 modifies the course of treatment based on the measured ECAP. In S120, alternating electric fields are applied to the target region during the course of treatment (e.g., when the AC voltage generator 40 applies an AC output to electrodes 45L/45R). In S130, the ECAP that is generated by the subject's body in response to the application of the alternating electric field in S120 is measured. This measurement is implemented using the ECAP electrodes 55 and the ECAP measurement system 50 as described above, and the measurements are reported to the controller 30. In S140, the controller 30 ascertains, based on the measured ECAP, if electrosensation is expected. If electrosensation is expected, the method continues at S160, in which the controller 30 sends commands to the control input of the signal generator 60, and those commands cause the signal generator 60 to generate the electrical signal that is configured (e.g., as described above) to reduce electrosensation. If electrosensation is not expected in S140, the method of FIG. 5 restarts from the beginning.

As described above in connection with FIG. 3, it is preferable to use an electric field whose orientation alternates between different directions in many anatomic locations using additional sets of electrode elements 45 (not shown in FIG. 4). In these circumstances, each set of electrode elements 45 preferably has its own associated set of ECAP electrodes 55 and its own associated set of electrodes 65 that operate similarly to the corresponding electrodes 55/65 depicted in FIG. 4 and described above.

In the example depicted in FIG. 4, the electrode elements 65 are adjacent to and distinct from the electrode elements 45. But in alternative embodiments, a single physical electrode element may serve as more than one of these electrode elements 45, 65 simultaneously (e.g., by combining two signals using superposition, and applying the superposed signal to a single electrode element), or a single physical electrode element may serve as more than one of these electrode elements 45, 65 at different times (e.g., using time division multiplexing).

The embodiments described above in connection with FIG. 2-5 rely on a passive array of ECAP electrodes to measure nerve activity, based on the theory that nerve activity can be used to determine that electrosensation is occurring or imminent. But a variety of alternative approaches to determine that electrosensation is occurring or imminent may be used instead of the ECAP-based techniques described above.

One example of an alternative approach uses electromyography signals to measure muscle activity, based on the theory that muscle activity (e.g., twitching) can be an indication that electrosensation is occurring. In these embodiments, the electromyography (EMG) signals are obtained using a set of EMG electrodes, pre-processed by an EMG system, and forwarded to a controller (which is similar to the controller 30 described above, but programmed to interpret EMG signals instead of ECAP signals). Another example of an alternative approach uses a mechanical sensor (e.g., an accelerometer) to measure muscle activity, based on the theory that muscle activity (e.g., twitching) can be an indication that electrosensation is occurring. In these embodiments, the vibration or acceleration signals are captured using the mechanical sensor, pre-processed by an appropriate front end, and forwarded to a controller (which is similar to the controller 30 described above, but programmed to interpret mechanical events instead of ECAP signals). Other approaches based on measured nerve or muscle activity can also be used.

In the embodiments described above in connection with FIG. 2 and FIG. 4, the AC voltage generator 40 generates AC outputs at a frequency between 50 kHz and 1 MHz. And when the output signal from the AC voltage generator 40 drives the electrode elements 45L, 45R an alternating electric field will be applied to the target region at a frequency between 50 kHz and 1 MHz. But this is not the only way to apply an alternating electric field with a frequency between 50 kHz and 1 MHz to the target region. To the contrary—alternative approaches for applying a 50 kHz-1 MHz alternating electric field to the target region may be employed.

One example of such an alternative approach relies on amplitude modulation (AM) concepts. More specifically, when a carrier signal at frequency f1 is AM modulated by a tone signal at frequency f2, the output of the modulator will include frequency components at f1, f1+f2, and f1−f2 (i.e., the original carrier, the sum, and the difference). Accordingly, if a MHz carrier is AM modulated by a 9.8 MHz tone signal, the output of the modulator will include frequency components at 200 kHz, 10 MHz, and 19.8 MHz. It therefore follows that if the output of the AM modulator is used to drive the electrode elements 45L, 45R, the 200 kHz component that is present in the output of the AM modulator will induce an alternating electric field in the target region with a frequency of 200 kHz (as well as additional frequency components in the MHz range).

In this AM modulation-based embodiment, the frequency of the alternating electric field that is applied to the target region can be increased based on the measured nerve or muscle activity by either increasing the carrier frequency or by decreasing the tone frequency; or the frequency of the alternating electric field that is applied to the target region can be decreased based on the measured nerve or muscle activity by either decreasing the carrier frequency or by increasing the tone frequency.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

1. A method of treating a target region of a subject's body with an alternating electric field, the method comprising:
applying an alternating electric field to the target region during a course of treatment, wherein the alternating electric field has a frequency between 50 kHz and 1 MHz;
measuring nerve or muscle activity that is generated by the subject's body in response to the application of the alternating electric field; and
modifying the course of treatment based on the measured nerve or muscle activity.

2. The method of claim 1, wherein the nerve or muscle activity comprises nerve activity and wherein the nerve activity is measured using a passive array of ECAP electrodes.

3. (canceled)

4. (canceled)

5. The method of claim 1, wherein the modifying comprises adjusting an amplitude of the alternating electric field that is applied to the target region based on the measured nerve or muscle activity.

6. The method of claim 1, wherein the modifying comprises reducing an amplitude of the alternating electric field that is applied to the target region when the measured nerve or muscle activity indicates that electrosensation is expected.

7. The method of claim 1, wherein the modifying comprises increasing an amplitude of the alternating electric field that is applied to the target region when the measured nerve or muscle activity indicates that the amplitude can be increased without causing electrosensation.

8. The method of claim 1, wherein the modifying comprises adjusting a frequency of the alternating electric field that is applied to the target region based on the measured nerve or muscle activity.

9. (canceled)

10. The method of claim 1, wherein the modifying comprises applying an electrical signal to the subject's body during each of a plurality of time intervals during the course of treatment,
wherein the electrical signal is configured to reduce electrosensation when the alternating electric field is applied during the course of treatment, and
wherein decisions of when to apply the electrical signal are based on the measured nerve or muscle activity.

11. The method of claim 10, wherein the decisions of when to apply the electrical signal are based on when the measured nerve or muscle activity indicates that electrosensation is expected.

12. The method of claim 11, wherein the electrical signal comprises a train of at least 10 pulses.

13. An apparatus for treating a target region of a subject's body with an alternating electric field, the apparatus comprising:
an AC voltage generator having an AC output that operates at a frequency between 50 kHz and 1 MHz and at least one control input; and
a controller configured to (a) accept signals from at least one sensor that measures nerve or muscle activity that is generated by the subject's body in response to the application of the alternating electric field and (b) modify a course of treatment based on the measured nerve or muscle activity.

14. The apparatus of claim 13, wherein the at least one sensor comprises a set of ECAP electrodes, and wherein the controller is configured to accept signals that represent nerve activity from the set of ECAP electrodes.

15. (canceled)

16. (canceled)

17. The apparatus of claim 13, further comprising at least one first electrode element configured for positioning on or in the subject's body and at least one second electrode element configured for positioning on or in the subject's body, wherein the AC output is applied between the at least one first electrode element and the at least one second electrode element.

18. The apparatus of claim 13, wherein the controller is programmed to send signals to the at least one control input that cause the AC voltage generator to adjust an amplitude of the AC output based on the measured nerve or muscle activity.

19. The apparatus of claim 13, wherein the controller is programmed to send signals to the at least one control input that cause the AC voltage generator to reduce an amplitude of the AC output when the measured nerve or muscle activity indicates that electrosensation is expected.

20. The apparatus of claim 13, wherein the controller is programmed to send signals to the at least one control input that cause the AC voltage generator to increase an amplitude of the AC output when the measured nerve or muscle activity indicates that the amplitude can be increased without causing electrosensation.

21. (canceled)

22. The apparatus of claim 13, wherein the controller is programmed to send signals to the at least one control input that cause the AC voltage generator to reduce a frequency of the AC output when the measured nerve or muscle activity indicates that electrosensation is expected.

23. The apparatus of claim 13, further comprising a signal generator that generates an electrical signal configured to reduce electrosensation when the alternating electric field is applied to the subject's body,
wherein the controller is programmed to activate the signal generator based on the measured nerve or muscle activity.

24. The apparatus of claim 23, wherein a decision to activate the signal generator is based on when the measured nerve or muscle activity indicates that electrosensation is expected.

25. The apparatus of claim 24, wherein the electrical signal comprises a train of at least 10 pulses.

26. The apparatus of claim 23, further comprising:
at least one first electrode element configured for positioning on or in the subject's body and at least one second electrode element configured for positioning on or in the subject's body, wherein the AC output is applied between the at least one first electrode element and the at least one second electrode element; and
a third electrode element configured for positioning on or in the subject's body and a fourth electrode element configured for positioning on or in the subject's body, wherein the electrical signal is applied between the third electrode element and the fourth electrode element.
